(19) United States
(12) Patent Application Publication
(10) Pub. No.: US 2025/0194592 A1
(43) Pub. Date: Jun. 19, 2025

(54) FATTY ACID DERIVATIVES FOR USE AS HERBICIDES

(71) Applicant: Clariant International Ltd, Muttenz (CH)

(72) Inventors: Peter BAUR, Schondorf (DE); Martin BAUER, Eppstein (DE); Felix HÖVELMANN, Mühldorf (DE); Luciana BODELON, Frankfurt am Main (DE); Javier CAMPOS CUEVAS, Bad Soden (DE); Stephanie GIESSLER, Frankfurt am Main (DE)

(73) Assignee: Clariant International Ltd, Muttenz (CH)

(21) Appl. No.: 19/064,820

(22) Filed: Feb. 27, 2025

Related U.S. Application Data
(62) Division of application No. 16/975,578, filed on Aug. 25, 2020.

(30) Foreign Application Priority Data
Feb. 26, 2018 (EP) 18158643.9

(51) Int. Cl. A01N 31/02; A01N 25/02

(52) U.S. Cl. CPC A01N 31/02; A01N 25/02

(57) ABSTRACT

Fatty acid derivatives of the formula (I)

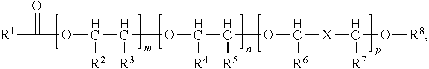

wherein
R¹ is an aliphatic group containing 1 to 17 carbon atoms, which is linear, branched, saturated or unsaturated;
R², R³, R⁴, R⁵, R⁶, R⁷ are, independently, hydrogen, methyl, ethyl or hydroxymethyl;
m, n and p are numbers from 0 to 17, with the proviso that m+n+p≥1, and m+n+p<18 where
   the different monomers can be arranged in statistical order, alternatingly or as a block copolymer and m, n and p can be a statistical mixture;
X is, independently, a covalent bond or hydroxy methylene;
R⁸ is an aliphatic group containing 1 to 10 carbon atoms, which is linear, branched, cyclic, saturated or unsaturated, substituted or unsubstituted phenyl or substituted or unsubstituted benzyl,
are useful as herbicides.

The invention relates to the use of certain fatty acid derivatives as herbicides, methods for controlling unwanted vegetation by applying certain fatty acid derivatives and new herbicidal fatty acid derivatives.

It is known that fatty acids and derivatives thereof can be used for the preparation of herbicidal compositions.

U.S. Pat. No. 6,503,869 B1 describes the addition of ammonium salts of fatty acids to post-emergent herbicidal compositions for the prevention or elimination of undesired vegetation.

U.S. Pat. No. 6,323,156 B1 describes aqueous herbicidal compositions which contain, as the active ingredient, an ammonium salt of a fatty acid wherein no more than 0.5 wt. % of the active ingredient is a free fatty acid.

U.S. Pat. No. 6,608,003 B2 describes aqueous herbicidal compositions based on ammonium salts of fatty acids enhanced by the addition of carboxylic diesters.

GB 2247621 A describes an aqueous herbicidal composition based on partially saponified fatty acids and a monohydric alcohol, having a synergistic effect on enhancing the rate and efficiency of mortality of herbage and/or undesirable flora.

WO 01/50862 A1 discloses a herbicidal composition containing a derivative of maleic hydrazide and a carboxylic acid component. The preferred composition describes an aqueous carboxylic ammonium salt compositions with saponification of the carboxylic acid as low as 25% and larger and a pH greater or equal to 6.

WO 2015/004086 A1 discloses herbicidal combinations of pelargonic acid and certain ALS inhibitors.

U.S. Pat. No. 6,383,585 B1 discloses herbicidal compositions containing a herbicidal fatty acid, such as pelargonic acid, and maleic hydrazide derivatives.

US 2013/0231247 discloses herbicidal compositions comprising pelargonic acid. WO 2011/161133 A2 discloses specific oil-in-water (EW) formulations of pelargonic acid.

The fatty acid formulations of the above described prior art have several disadvantages. During the production, they require the handling of concentrated and corrosive alkaline solutions to neutralize the fatty acid. Aqueous solutions of fatty acid ammonium salts are limited in their maximum fatty acid content and generally corrosive. Moreover, in open environment the evaporation and release of ammonia will generate not only an unpleasant offensive smell but also release volatile free-fatty acid from its ammonium salt. Moreover, the low water-solubility of the free fatty acid may negatively affect the biological performance of the product.

U.S. Pat. No. 5,106,410 includes a ready-to-use herbicidal emulsion including a fatty acid, a surfactant component, preferably comprising at least one quaternary ammonium salt and a balance of water. It also features a concentrate composition having a fatty acid and one or more hydrophobic surfactants. The most preferred surfactants for use with this concentrate composition are those which lack a terminal group, such as a hydroxyl group, which are reactive with the fatty acid component.

U.S. Pat. No. 5,035,741 describes a herbicidal composition, suitable for emulsification in water, containing a monocarboxylic acid component, an emulsifier component and an oil component selected from the group consisting of triglycerides, terpenoids and paraffinic mineral oils. These compositions aim at providing an environmentally compatible herbicide with reduced eye and skin corrosivity.

The use of surfactants based on polyoxyethylene together with free pelargonic acid as herbicidal compositions is disclosed in JP 2011001337 A.

All these compositions of the prior art contain free fatty acids and show one or more of the following disadvantages: 1. handling of concentrated ammonia or other alkaline substances, 2. unpleasant odor due to high volatility of free fatty acid, 3. limited active ingredient content (giving high product volumes), 4. limited biological efficacy, a.o. due to lack of suitable adjuvants which boost the biological performance and low water-solubility of the free fatty acid, 5. eye and skin irritation properties and/or 6. selected choice of surfactant and emulsifier e.g. due to incompatibility to the fatty acid component.

US 2016/0102271 discloses unsaturated alkoxylated fatty esters and derivates as components of agrochemical compositions. US 2016/0168041 A1 and U.S. Pat. No. 8,946,122 B2 disclose di- and tripropylene glycol methyl ether acetates for use in agrochemical compositions. U.S. Pat. No. 4,975,113 discloses tridecanoic acid 2-methoxyethyl ester for use in agrochemical compositions. The use of hexadecanoic acid 2-(2-methoxy-ethoxyl)ethyl ester in agrochemical compositions is disclosed in JP-H08 157 819 A. JP 2014 2018 482 A and JP 6 279 804 B1 disclose fatty acid polyoxyalkylene alkyl ethers as components of bactericidal and agrochemical compositions respectively.

U.S. Pat. No. 5,284,819 discloses a herbicidal activity of monoglycol esters of fatty acids such as pelargonic acid.

However, there is still a need to provide improved fatty acid based compounds which exhibit excellent herbicidal and/or dessicant efficacy and address the disadvantages of the compounds of the prior art.

It has now been found that esters of certain fatty acids, preferably end-capped, with alkylene glycol and/or glycerol mono-, oligo- or polymers show excellent herbicidal activity while avoiding the problems of the prior art compounds.

Accordingly, the invention provides the use of one or more fatty acid derivatives of the formula (I)

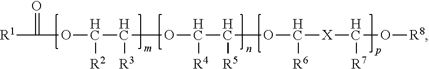

wherein
R¹ is an aliphatic group containing 1 to 17 carbon atoms, which is linear, branched, saturated or unsaturated;
R², R³, R⁴, R⁵, R⁶, R⁷ are, independently, hydrogen, methyl, ethyl or hydroxymethyl;
m, n and p are numbers from 0 to 17, with the proviso that m+n+p≥1, preferably >1, and m+n+p<18 where
the different monomers can be arranged in statistical order, alternatingly or as a block copolymer and m, n and p can be a statistical mixture;
X is, independently, a covalent bond or hydroxy methylene;
R⁸ is hydrogen or an aliphatic group containing 1 to 10 carbon atoms, which is linear, branched, cyclic, saturated or unsaturated, substituted or unsubstituted phenyl or substituted or unsubstituted benzyl, preferably an aliphatic group containing 1 to 10 carbon atoms, which is linear, branched, cyclic, saturated or unsaturated, substituted or unsubstituted phenyl or substituted or unsubstituted benzyl, as herbicides.

In a further embodiment of the invention there is provided a method for controlling unwanted vegetation or plant growth, comprising the step of applying one or more fatty acid derivatives of the formula (I) to the unwanted vegetation.

In a further embodiment of the invention there is provided a herbicidal composition comprising one or more fatty acid derivatives of the formula (I).

The fatty acid derivatives of formula (I) are partly new and partly known. Accordingly, in a further embodiment of the invention there are provided fatty acid derivatives of the formula (I)

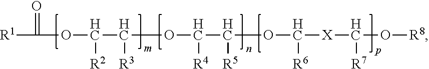

wherein
R¹ is an aliphatic group containing 1 to 17 carbon atoms, which is linear, branched, saturated or unsaturated;
R², R³, R⁴, R⁵, R⁶, R⁷ are, independently, hydrogen, methyl, ethyl or hydroxymethyl;
m, n and p are numbers from 0 to 17, with the proviso that m+n+p≥1, and m+n+p<18 where
the different monomers can be arranged in statistical order, alternatingly or as a block copolymer and n, m and p can be a statistical mixture;
X is, independently, a covalent bond or a hydroxy methylene group;
R⁸ is an aliphatic group containing 1 to 10 carbon atoms, which is linear, branched, cyclic, saturated or unsaturated, substituted or unsubstituted phenyl or substituted or unsubstituted benzyl.

In a preferred embodiment of the invention there are provided fatty acid derivatives of the formula (I)

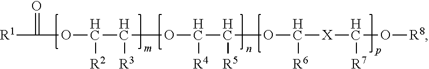

wherein
R¹ is an alkyl group containing 5 to 13 carbon atoms, which is linear or branched;
R², R³ are, independently, hydrogen, methyl, ethyl or hydroxymethyl with the proviso that one of R² and R³ is hydrogen and the other is different from hydrogen;
R⁴, R⁵ are hydrogen;
m, n are numbers from 0 to 12, with the proviso that m+n>4, and m+n<12 where the different monomers can be arranged in statistical order, alternatingly or as a block copolymer;
p is 0 and
R⁶ is a methyl group.

In summary, with this invention it has surprisingly been found that herbicidal esters of fatty acids with alkylene glycol or glycerol mono-, oligo- or polymers are stable and have a lower skin and eye irritation potential and that these compounds exhibit superior contact herbicide- and/or dessicant activity and improved rainfastness. As a further advantage it has been found that the compounds of the invention (and diluted aqueous compositions thereof) are far less volatile and do not unpleasantly smell in comparison to products containing fatty acids such as pelargonic acid or ammonia neutralized herbicidal fatty acid formulations after application.

The term “herbicide” as used herein defines an agent that shows activity in the control of unwanted vegetation and/or the regulation of plant growth as Plant Growth Regulator (PGR). Likewise, the term “herbicidal” as used herein means showing activity in the control of unwanted vegetation and/or the regulation of plant growth.

In formula (I) alkyl radicals having more than two carbon atoms can be straight-chain or branched. Alkyl radicals are, for example, methyl, ethyl, n- or isopropyl, n-, iso-, t- or 2-butyl, pentyls, hexyls, such as n-hexyl, isohexyl and 1,3-dimethylbutyl.

Depending on the nature and the attachment of the substituents, the fatty acid derivatives of the formula (I) may be present as stereoisomers. If, for example, one or more asymmetrically substituted carbon atoms are present, there may be enantiomers and diastereomers. Stereoisomers may be obtained from the mixtures resulting from the preparation using customary separation methods, for example by chromatographic separation techniques. It is also possible to prepare stereoisomers selectively by using stereoselective reactions employing optically active starting materials and/or auxiliaries. The invention also relates to all stereoisomers and mixtures thereof embraced by the formula (I) but not specifically defined.

R¹ is preferably straight-chain or branched, preferably straight-chain, (C₁-C₁₇)-alkyl, straight-chain or branched, preferably straight-chain, (C₂-C₁₇)-alkenyl or alkadienyl, or straight-chain or branched, preferably straight-chain, (C₂-C₁₇)-alkynyl, more preferably alkyl.

R⁸ is preferably straight-chain or branched, preferably straight-chain, (C₁-C₁₀)-alkyl, straight-chain or branched, preferably straight-chain, (C₂-C₁₀)-alkenyl or alkadienyl, or straight-chain, branched, preferably straight-chain, (C₂-C₁₇)-alkynyl, (C₃-C₅)-cycloalkyl, preferably (C₅-C₆)-cycloalkyl, substituted or, preferably, unsubstituted phenyl, or substituted or, preferably unsubstituted, benzyl, where substituted preferably means substitution with one or more, preferably one or two groups selected from (C₁-C₄)-alkyl, (C₁-C₄)-alkoxy and halogen. More preferably R⁸ is alkyl, phenyl or benzyl, in particular alkyl.

In a preferred embodiment of the fatty acid derivatives of the formula (I)
R¹ contains 5 to 15 carbon atoms, more preferably 5 to 13 carbon atoms, in particular 7 to 11 carbon atoms, and is preferably linear or branched alkyl, in particular linear alkyl.

In a further preferred embodiment R¹ is n-octyl.

In a further preferred embodiment
R⁸ is an alkyl group containing 1 to 10 carbon atoms, preferably 1 to 5 carbon atoms, in particular 1 to 3 carbon atoms, in particular 1 to 3 carbon atoms, and is preferably a linear alkyl group.

In a further preferred embodiment R⁸ is methyl.

In a further preferred embodiment R² is H and R³ is H.

In a further preferred embodiment R³ is hydroxymethyl.

In a further preferred embodiment m is a number from 1 to 10, more preferred 3 to 7.

In a further preferred embodiment n is 0 and p is 0.

In a further preferred embodiment R¹ is a linear alkyl group with 7 to 9 carbon atoms; R² and R³ are H; n and p are 0; m is a number >4, preferably ≥5 to ≤9, preferably 7, and
R⁸ is a methyl group.

In a further preferred embodiment p is 0 and m and n are independently numbers from 0 to 12, with the proviso that m+n>4, more preferably m+n≥5, and <12, more preferably <9, in particular ≤7.

The term number as used herein means 0 or a positive rational number. n and m are statistical values, therefore the monomer units m and n can be statistical mixtures.

Preference is further given to compounds of the formula (I) in which
R¹ is an aliphatic group, preferably an alkyl group, containing 5 to 15, more preferred 7 to 10, in particular 8 carbon atoms;
R², R³, R⁴, R⁵, R⁶, R⁷ is H or methyl, more preferred H;
m is a number from 0 to 15, more preferred 1 to 10, in particular 0 to 3;
n is a number from 0 to 15, more preferred 0 to 5, in particular 1 to 5;
p is a number from 0 to 15, more preferred 1 to 10, in particular 1 to 5;
with the proviso that m+n+p is ≥1 and m+n+p<18, preferably <15;
X is a covalent bond and
R⁸ is an aliphatic group, preferably an alkyl group, containing 1 to 4 carbon atoms, where propyl and butyl may be linear or branched, and more preferably methyl.

Further preference is given to fatty acid derivatives off the formula (I), wherein
R¹ is n-octyl;
R² and R³ are H;
R⁸ is methyl;
m a number <4 and >12, and
n and pare 0.

Further preference is given to a fatty acid derivative of the formula (I),
wherein
R¹ is n-octyl;
R² and R³ are H;
R⁸ methyl;
m 6 and;
n and pare 0.

Particularly preferred are the fatty acid derivatives of formula (I) A1 to A16 disclosed in the Examples.

The compounds of the invention used for controlling unwanted vegetation according to the invention are preferably derived from fatty acids which show herbicidal activity and can be one herbicidal fatty acid and/or a mixture of herbicidal fatty acids. Esters of fatty acids (as defined herein) which may preferably be used include esters of carboxylic acids comprising 2 to 18 carbon atoms, especially caprylic acid, pelargonic acid, capric acid, undecanoic acid, 10-undecenoic acid, lauric acid, myristic acid, palmitic acid, oleic acid and mixtures thereof. Esters of other fatty acid mixtures such as soybean fatty acids and coconut fatty acids and other naturally occurring fatty acid mixtures may also form the fatty acid component used in the invention. Exemplary esters of fatty acids are esters of pelargonic acid or C8/10 or C12/14. Most preferably esters of pelargonic acid are used in the invention.

The compounds of the invention used for controlling unwanted vegetation are esters of the above mentioned fatty acids with alkylene glycol or glycerol mono-, oligo- or polymers having from 1 to <18 repeating units of alkylene glycol or glycerol. In a preferred embodiment, the number of alkylene glycol or glycerol repeating units is from 1 to 15. In an even more preferred embodiment, the number of alkylene glycol or glycerol repeating units is from 1 to 7. In a particularly preferred embodiment, the number of alkylene glycol or glycerol repeating units is from 3 to 7.

The alkylene glycol is selected from the group consisting of ethylene glycol, 1,2-propylene glycol, 1,3-propylene glycol, 1,2-butylene glycol and 1,3-butylene glycol. In a preferred embodiment the alkylene glycol is ethylene glycol or 1,2-propylene glycol. In an even more preferred embodiment the alkylene glycol is ethylene glycol.

The end group of the alkylene glycol or glycerol mono-, oligo or polymer chain that is not esterified with the above mentioned fatty acid carries a terminal functional group which is preferably an ether group. If glycerol mono-, oligo or polymer chains are employed, the remaining hydroxyl groups are, independently, unmodified or preferably transformed into ether groups.

The ether group is an ether of the alkylene glycol mono-, oligo- or polymer with a linear, branched, cyclic, benzylic or aromatic alcohol comprising 1 to 10 carbon atoms. Preferably, the alcohol comprises 1 to 6 carbon atoms. Even more preferably, the alcohol comprises 1 carbon atom.

In one embodiment of the invention, the compound of the invention is an ester of pelargonic acid with an ethylene glycol oligomer comprising 5 or 6, preferably 6, ethylene glycol repeating units, which is terminated with an ether group derived from decane-1-ol or methanol, preferably methanol.

In another embodiment, the compound of the invention is an ester of pelargonic acid with an ethylene glycol oligomer comprising 3 ethylene glycol repeating units, which is terminated with an ether group derived from hexane-1-ol or methanol, preferably methanol.

In a preferred embodiment, the compound of the invention is an ester of pelargonic acid with an ethylene glycol polymer comprising ethylene glycol repeating units and terminated with an ether group derived from methanol.

The fatty acid derivatives of the formula (I) can be prepared by methods know to those skilled in the art, as described e.g. in U.S. Pat. No. 7,595,291 B2 (BASF SE, Esterified alkyl alkoxylates used as low-foam surfactants). The compounds are usually prepared by condensation of fatty acid or fatty acid ester and the respective alcohol alkoxylate by removal of water or the alcohol, respectively, in the presence of an acidic catalyst. Alcohol alkoxylate derivatives are prepared by reacting a suitable precursor, e.g. an alcohol or and alkoxylated alcohol, with an alkylene oxide in the presence of an alkoxylation catalyst. Among others, NaOMe, KOMe, NaOH, KOH, alkaline earth-based catalysts or double metal cyanide (DMC) catalysts can be used (e.g. SHELL OIL COMPANY—US2012/310004, 2012, A1 Nonyl alcohols with a low degree of branching and their derivatives). The composition of the alkylene oxide chain can be either a single pure alkylene oxide, preferably selected from the group of ethylene oxide, propylene oxide or butylene oxide, or a copolymer of a binary or ternary mixture of alkylene oxides. The copolymers may be arranged in a statistical distribution, alternatingly, as block copolymers or a mixture thereof.

Compounds of comparable chemical compositions can be realized by reacting a carboxylic acid ester with one or more alkylene oxides in the presence of a suitable insertion catalyst. The ester is preferably, but not exclusively, a methyl ester. Specific procedures are disclosed, e.g., in Scholz H. J., Stuhler H., Quack J., Schuler W., Trautmann, M. (1988) Verfahrung zur Herstellung von Carbonsäureestern von Alkylenglykolethern und deren Verwendung, DE 3810793A1 (Hoechst), Weerasooriya U, Robertson D T, Lin J, Leach B E, Aeschbacher C L, Sandoval T S (1995) Process for alkoxylation of esters and products produced therefrom, U.S. Pat. No. 5,386,045, and Tanaka T, Imamaka T, Kaeaguchi T, Nagumo H (1997) Process for producing ester alkoxide compound and surfactant comprising ester alkoxylate compound, EP0783012.

Use can be further made of the detailed instructions in the examples section which describe in detail how to prepare these and any further compounds of the invention.

The compounds according to the invention can be used in undiluted form or, e.g. as solutions, emulsions, emulsifiable concentrates, sprayable solutions, gels, dusting products or granules in customary formulations. The invention therefore also provides herbicidal compositions which comprise one or more compounds according to the invention. Preferably, the compositions of the invention comprise one or more compounds according to the invention and one or more formulation aids.

The compounds according to the invention can be formulated in various ways according to which biological and/or physicochemical parameters are required. Possible formulations include, for example: soluble liquids (SL), emulsions (EW) such as oil-in-water and water-in-oil emulsions, microemulsions (ME), sprayable solutions, suspension concentrates (SC), sulspoemulsions (SE), other oil-, (poly)glycol-, glycerol-based, optionally water containing dispersions, oil-miscible solutions (OF), wettable powders (WP), water-soluble powders (SP), water-soluble concentrates, emulsifiable concentrates (EC), capsule suspensions (CS), dusting products (DP), seed-dressing products, granules for scattering and soil application, granules (GR) in the form of microgranules, granules for scattering and soil application, granules (GR) in the form of microgranules, spray granules, coated granules and adsorption granules, water-dispersible granules (WG), water-soluble granules (SG), ULV formulations, micro-capsules and waxes. The compounds according to the invention can also be offened as AL type, which includes undiluted pure product or so called ready-to-use preparations. These individual types of formulations are known in principle and are described, for example, in: Winnacker-Kuchler, “Chemische Technologie” [Chemical technology], Volume 7, C. Hanser Verlag Munich, 4^{th} Ed. 1986, Wade van Walkenburg, “Pesticide Formulations”, Marcel Dekker, N.Y., 1973; K. Martens, “Spray Drying” Handbook, 3^{rd} Ed. 1979, G. Goodwin Ltd. London. Apart from any conventional application system an application by drones is feasible.

The necessary formulation aids, such as inert materials, surfactants, solvents and further additives, are likewise known and are described, for example, in: Watkins, “Handbook of Insecticide Dust Diluents and Carriers”, 2^{nd} Ed., Darland Books, Caldwell N.J., H. v. Olphen, “Introduction to Clay Colloid Chemistry”; 2^{nd} Ed., J. Wiley & Sons, N.Y.; C. Marsden, “Solvents Guide”; 2^{nd} Ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964, Schönfeldt, “Grenzflächenaktive Äthylenoxidaddukte” [Interface-active ethylene oxide adducts], Wiss. Verlagsgesell., Stuttgart 1976; Winnacker-Küchler, “Chemische Technologie”, Volume 7, C. Hanser Verlag Munich, 4^{th} Ed. 1986.

The composition according to the invention may further comprise one or more suitable emulsifier components enabling an emulsion, e.g. an oil in water emulsion, to be formed when the composition of the invention is added to water.

Preferably, the emulsifier component is at least one non-ionic surfactant selected from the group of alkoxylated alcohols, ethoxylated alcohols, ethopropoxylated alcohols, alkylphenolethoxylates, alkoxylated tristyrylphenols, alkoxylated tributylphenols, alkylaminethoxylates, ethoxylated vegetable oils including their hydrogenates, polyadducts of ethylene oxide and propylene oxide (e.g. polyoxyethylene-polyoxypropylene block copolymers and their derivatives), ethoxylated fatty acids, nonionic polymeric surfactants (e.g. polyvinylalcohol, polyvinylpyrrolidone, poly-methacrylates and their derivatives), sorbitan esters and their ethoxylates, sorbitol-esters, propylene glycol esters of fatty acids, alkylpolyglycosides, glucamides and polyglycerolesters.

Examples of especially preferred non-ionic surfactants are ethoxylated alcohols (e.g. Brij 020-SO-(MV), Croda), ethopropoxylated alcohols (e.g. Agnique KE 3551, BASF), alkoxylated tristyrylphenols, ethoxylated tristyrylphenols (e.g. Soprophor TS/16, Rhodia), ethopropoxylated tristyrylphenols (e.g. Soprophor 796/P, Rhodia) and ethoxylated vegetable oils (e.g. Tanemul® KS, Tanatex Chemicals), glucamides (Synergen GA, Clariant).

The composition according to the invention may also comprise—as an additional emulsifier component—an anionic surfactant as a salt of a multivalent cation, e.g. calcium. Examples of such anionic surfactants are calcium salts of alkylarylsulfonates CALSOGEN® 4814 (Clariant), NANSA EVM 70/2E (Huntsmann) and Emulsifier 1371 A (Lanxess).

The composition of the invention may further comprise one or more organic solvents. In combination with the other components, the solvent should give preferably a homogeneous and even more preferably a clear composition with good emulsifying properties upon dilution into water.

A suitable organic solvent can be chosen from the group of organic water-unsoluble or water soluble solvents. Organic water-unsoluble solvents are preferably selected from the group consisting of aromatic hydrocarbons, aliphatic hydrocarbons, fatty acid dimethylamides, carboxylic acid esters, alcohols, polyalkylene glycols, esters of plant oils, glycerol ester oils and mixtures thereof. Water soluble solvents are, e.g., alcohols.

Aromatic and aliphatic hydrocarbons such as hexane, cyclohexane, benzene, toluene, xylene, mineral oil or kerosin or substituted naphthalenes, mixtures of mono- and polyalkylated aromatics are commercially available under the registered trademarks Solvesso®, Shellsol®, Petrol Spezial®, Plurasolv® and Exxsol®.

Esters of plant oils, which may be used as non-polar, water-immiscible solvents according to the present invention are, as a rule, alkyl esters obtainable from medium chained fatty acids by esterification with alkanols or by transesterification of the corresponding plant oils preferably in the presence of a lipase.

Glycerol ester oils are to be understood as meaning esters of saturated or unsaturated fatty acids with glycerol. Mono-, di- and triglycerides, and their mixtures, are suitable. Preference is given to fatty acid triglycerides.

The solvent for the compositions according to the invention is preferably selected from the group consisting of C₁-C₄ alkyl ester (preferably methyl ester) of a C₅-C₂₀ (preferably C₉-C₁₈) saturated or unsaturated fatty acid or a mixture of such esters and C₆-C₂₀-fatty acids mono-, di- and/or triglycerides.

Commercial formulations of such esters include Witconol® 1095 and Witconol® 2309 (methyl esters of plant oils, available from Witco Corporation), Emery 2219 (58% methyl oleate, 24% methyl stearate, 14% methyl linoleate, 4% methyl palmitate), Emerest 2301 (76% methyl oleate, 24% methyl esters of other C₁₄-C₁₈ fatty acids), Emery 2270 (70% methyl laurate, 28% methyl myristate, 1% methyl palmitate), and Emery 2209 (55% methyl caprylate, 40% methyl caprate, 3% methyl caproate, 2% methyl laurate) all available from Henkel Corporation, Emery Group; Stepan C₂₅ (methyl caprylate+methyl caprate), available from Stepan Company; KE-1870 (methyl oleate) and CE-810 (methyl caprylate+methyl caprate), available from Proctor & Gamble Company, Priolube 1400 (methyl oleate), available from Unichema; PAMAK W4 (tall oil fatty acids), available from Hercules Inc.; ACTINOL FAI and D30LR (tall oil fatty acids), available from Arizona Chemical Company; Kemester EX-1550 (methyl ester of polyoverized tall oil), Kemester 3695 (methyl ester of dimer acid), and Witconol 2301 methyl oleate, all available from Witco Corporation; Agnique ME 18 RD-F, available from BASF; and Synative ES ME SU (methyl ester of rapeseed oil fatty acid, also known as methyl canolate) available from BASF, octanoyl glyceride/decanoyl glyceride mixture Miglyol® 812 from Sasol.

Other suitable organic solvents which may be employed in the compositions according to the invention may be water-soluble. They are preferably selected from the group consisting of water-soluble alcohols such as glycerins and propylenglycol, polyalkylene glycols, alkylene carbonates and carboxylic acid esters (eg. citric acid esters, dibasic esters and lactate esters), alkylpyrrolidons (N-Methylpyrrolidone, N-butylpyrrolidone), methyl-5-(dimethylamino)-2-methyl-5-oxopentanoate (Rhodiasolv Polarclean), DMSO and lactones.

The content of the optional organic solvent in the composition according to the invention is preferably 0% to 90% by weight, more preferably 5% to 60% by weight and most preferably between 10% to 50% by weight.

In a preferred embodiment the composition according to the invention does not contain an organic solvent.

The herbicidal compositions according to the invention contain generally from 2 to 99.9% by weight, in particular from 2 to 99% by weight, of compounds according to the invention. In wettable or soluble powders, the active compound concentration is, for example, from about 10 to 90% by weight, the remainder to 100% by weight consisting of customary formulation aids. In the case of emulsifiable concentrates, the active compound concentration can be from about 2 to 90, preferably from 5 to 80, % by weight. Formulations in the form of dusts comprise from 2 to 60% by weight of active compound, preferably usually from 5 to 40% by weight of active compound; sprayable solutions contain from about 1 to 80% by weight, preferably from 2 to 50% by weight of active compound. In the case of water-dispersible granules, the active compound content depends partially on whether the active compound is present in liquid or solid form and on which granulation auxiliaries, fillers, etc., are used. In the water-dispersible granules, the content of active compound is, for example, between 2 and 95% by weight, preferably between 10 and 80% by weight.

In addition, the active compound formulations mentioned optionally comprise the respective customary adhesives, wetting agents, dispersants, emulsifiers, synergists, penetrants, preservatives, antifreeze agents and solvents, fillers, carriers and dyes, defoamers, evaporation inhibitors and agents which influence the pH and the viscosity.

Based on these formulations, it is also possible to produce combinations with other pesticidally active compounds, such as, for example further herbicides, insecticides, acaricides, fungicides, and also with safeners, fertilizers and/or growth regulators, for example in the form of a finished formulation or as a tank mix.

Components which can be used in combination with the compositions according to the invention in mixed formulations or in the tank mix are, for example, known active compounds as they are described, for example, in Weed Research 26, 441-445 (1986), or “The Pesticide Manual”, 17^{th} edition, The British Crop Protection Council and the Royal Soc. of Chemistry, 2015 and adjuvants as described in “Compendium of adjuvants for herbicides” (www.herbicide-adjuvants.com).

Examples of active compounds which may be mentioned as herbicides or plant growth regulators which are known from the literature and which can be combined with the compositions according to the invention are the following:

Acetochlor, acibenzolar, acibenzolar-s-methyl, acifluorfen, acifluorfen-sodium, aclonifen, alachlor, allidochlor, alloxydim, alloxydim-sodium, ametryn, amicarbazone, amidochlor, amidosulfuron, aminopyralid, amitrole, ammoniumsulfamat, ancymidol, anilofos, asulam, atrazine, azafenidin, azimsulfuron, aziprotryn, BAH-043, BAS-140H, BAS-693H, BAS-714H, BAS-762H, BAS-776H, BAS-800H, beflubutamid, benazolin, benazolin-ethyl, bencarbazone, benfluralin, benfuresate, bensulide, bensulfuron-methyl, bentazone, benzfendizone, benzobicyclon, benzofenap, benzofluor, benzoylprop, bifenox, bilanafos, bilanafos-sodium, bispyribac, bispyribac-sodium, bromacil, bromobutide, bromofenoxim, bromoxynil, bromuron, buminafos, busoxinone, butachlor, butafenacil, butamifos, butenachlor, butralin, butroxydim, butylate, cafenstrole, carbetamide, carfentrazone, carfentrazone-ethyl, chlomethoxyfen, chloramben, chlorazifop, chlorazifop-butyl, chlorbromuron, chlorbufam, chlorfenac, chlorfenac-sodium, chlorfenprop, chlorflurenol, chlorflurenol-methyl, chloridazon, chlorimuron, chlorimuron-ethyl, chlormequat-chlorid, chlomitrofen, chlorophthalim, chlorthal-dimethyl, chlorotoluron, chlorsulfuron, cinidon, cinidon-ethyl, cinmethylin, cinosulfuron, clethodim, clodinafop clodinafop-propargyl, clofencet, clomazone, clomeprop, cloprop, clopyralid, cloransulam, cloransulam-methyl, cumyluron, cyanamide, cyanazine, cyclanilide, cycloate, cyclosulfamuron, cycloxydim, cycluron, cyhalofop, cyhalofop-butyl, cyperquat, cyprazine, cyprazole, 2,4-D, 2,4-DB, daimuron/dymron, dalapon, daminozide, dazomet, n-decanol, desmedipham, desmetryn, detosyl-pyrazolate (DTP), diallate, dicamba, dichlobenil, dichlorprop, dichlorprop-p, diclofop, diclofop-methyl, diclofop-p-methyl, diclosulam, diethatyl, diethatyl-ethyl, difenoxuron, difenzoquat, diflufenican, diflufenzopyr, diflufenzopyr-sodium, dimefuron, dikegulac-sodium, dimefuron, dimepiperate, dimethachlor, dimethametryn, dimethenamid, dimethenamid-p, dimethipin, dimetrasulfuron, dinitramine, dinoseb, dinoterb, diphenamid, dipropetryn, diquat, diquat-dibromide, dithiopyr, diuron, DNOC, eglinazine-ethyl, endothal, eptc, esprocarb, ethalfluralin, ethametsulfuron-methyl, ethephon, ethidimuron, ethiozin, ethofirmesate, ethoxyfen, ethoxy fen-ethyl, ethoxysulfirron, etobenzanid, F-5331, e. N-[2-chlor-4-fluor-5-[4-(3fluorpropyl)-4,5-dihydro-5-oxo-IH-tetrazol-I-yl]-phenyl]-ethansulfonamid, fenoprop, fenoxaprop, fenoxaprop-p, fenoxaprop-ethyl, fenoxaprop-p-ethyl, fentrazamide, fenuron, flamprop, flamprop-m-isopropyl, flamprop-m-methyl, flazasulfuron, florasulam, fluazifop, fluazifop-p, fluazifop-butyl, fluazifop-p-butyl, fluazolate, flucarbazone, flucarbazone-sodium, flucetosulfuron, fluchloralin, flufenacet (thiafluamide), flufenpyr, flufenpyr-ethyl, flumetralin, flumetsulam, flumiclorac, flumiclorac-pentyl, flumioxazin, flumipropyn, fluometuron, fluorodifen, fluoroglycofen, fluoroglycofen-ethyl, flupoxam, flupropacil, flupropanate, flupyrsulfuron, flupyrsulfuron-methyl-sodium, flurenol, flurenol-butyl, fluridone, flurochloridone, fluroxypyr, fluroxypyr-meptyl, flurprimidol, flurtamone, fluthiacet, fluthiacet-methyl, fluthiamide, fomesafen, foramsulfuron, forchlorfenuron, fosamine, furyloxyfen, gibberellinic acid, glufosinate, 1-glufosinate, 1-glufosinate-ammonium, glufosinate-ammonium, glyphosate, glyphosate-isopropylammonium, H-9201, halosafen, halosulfuron, halosulfuron-methyl, haloxyfop, haloxyfop-p, haloxyfop-ethoxyethyl, haloxyfop-p-ethoxy ethyl, haloxyfop-methyl, haloxyfop-p-methyl, hexazinone, hnpc-9908, HOK-201, HW-02, imazamethabenz, imazamethabenz-methyl, imazamox, imazapic, imazapyr, imazaquin, imazethapyr, imazosulfuron, inabenfide, indanofan, indolacetic acid (IAA), 4-indol-3-yl-butanoic acid (IBA), iodosulfuron, iodosulfuron-methyl-sodium, ioxynil, isocarbamid, isopropalin, isoproturon, isouron, isoxaben, isoxachlortole, isoxaflutole, isoxapyrifop, IDH-100, KUH-043, KUH-071, karbutilate, ketospiradox, lactofen, lenacil, linuron, maleinic acid hydrazid, MCPA, MCPB, MCPB-methyl, -ethyl und-sodium, mecoprop, mecoprop-sodium, mecoprop-butotyl, mecoprop-p-butotyl, mecoprop-p-dimethylammonium, mecoprop-p-2-ethylhexyl, mecoprop-p-kalium, mefenacet, mefluidide, mepiquat-chlorid, mesosulfuron, mesosulfuron-methyl, mesotrione, methabenzthiazuron, metam, metamifop, metamitron, metazachlor, methazole, methoxyphenone, methyldymron, 1-methylcyclopropen, methylisothiocyanat, metobenzuron, metobenzuron, metobromuron, metolachlor, s-metolachlor, metosulam, metoxuron, metribuzin, metsulfuron, metsulfuron-methyl, molinate, monalide, monocarbamide, monocarbamide-dihydrogensulfat, mono linuron, monosulfuron, monuron, MT 128, MT-5950, i.e. N-[3-chlor-4-(1-methylethyl)-phenyl]-2-methylpentanamide, NGGC-011, naproanilide, napropamide, naptalam, NC-310, i.e. 4-(2,4-dichlorobenzoyl)-I-methyl-5-benzyl-oxypyrazole, neburon, nicosulfuron, nipyraclofen, nitralin, nitrofen, nitrophenolat-sodium (mixture of isomers), nitrofluorfen, nonanoic acid, norflurazon, orbencarb, orthosulfamuron, oryzalin, oxadiargyl, oxadiazon, oxasulfuron, oxaziclomefone, oxyfluorfen, paclobutrazol, paraquat, paraquat-dichlorid, pelargonic acid (nonanoic acid), pendimethalin, pendralin, penoxsulam, pentanochlor, pentoxazone, perfluidone, pethoxamid, phenisopham, phenmedipham, phenmedipham-ethyl, picloram, picolinafen, pinoxaden, piperophos, pirifenop, pirifenop-butyl, pretilachlor, primisulfuron, primisulfuron-methyl, probenazole, profluazol, procyazine, prodiamine, prifluraline, profoxydim, prohexadione, prohexadione-calcium, prohydrojasmone, prometon, prometryn, propachlor, propanil, propaquizafop, propazine, propham, propisochlor, propoxycarbazone, propoxycarbazone-sodium, propyzamide, prosulfalin, prosulfocarb, prosulfuron, prynachlor, pyraclonil, pyraflufen, pyraflufen-ethyl, pyrasulfotole, pyrazolynate (pyrazolate), pyrazosulfuron-ethyl, pyrazoxyfen, pyribambenz, pyribambenz-isopropyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-p, quizalofop-p-ethyl, quizalofop-p-tefuryl, rimsulfuron, secbumeton, sethoxydim, siduron, simazine, simetryn, SN-106279, sulcotrione, sulfallate (cdec), sulfentrazone, sulfometuron, sulfometuron-methyl, sulfosate (glyphosate-trimesium), sulfosulfuron, SYN-523, SYP-249, SYP-298, SYP-300, tebutam, tebuthiuron, tecnazene, tefuryltrione, tembotrione, tepraloxydim, terbacil, terbucarb, terbuchlor, terbumeton, terbuthylazine, terbutryn, th-547, thenylchlor, thiafluamide, thiazafluron, thiazopyr, thidiazimin, thidiazuron, thiencarbazone, thiencarbazone-methyl, thifensulfuron, thifensulfuron-methyl, thiobencarb, tiocarbazil, topramezone, tralkoxydim, triallate, triasulfuron, triaziflam, triazofenamide, tribenuron, tribenuron-methyl, trichlor acetic acid (tea), triclopyr, tridiphane, trietazine, trifloxysulfuron, trifloxysulfuron-sodium, trifluralin, triflusulfuron, triflusulfuron-methyl, trimeturon, trinexapac, trinexapac-ethyl, tritosulfuron, tsitodef, uniconazole, uniconazole-p, vemolate, ZJ-0166, ZJ-0270, ZJ-0543 and ZJ-0862.

Common names are used in accordance with the International Organization for Standardization (ISO) or the chemical names, if appropriate together with a customary code number, of the compounds and always comprise all applicable forms such as acids, salts, ester, or modifications such as isomers, like stereoisomers and optical isomers.

Preferred herbicides for application in combination with the compounds of the invention are one or more herbicides selected from:
a) sulfonyl ureas and sulfonyl amino carbonyl-triazolinones,
b) auxins (synthetic and esters),
c) botanical oils, from non-volatile vegetable oils like linseed oil to volatile essential oils, e.g. terpenes, clove oil,
d) glyphosate, glufosinate,
e) maleic hydrazide,
f) Fe-chelates,
g) benzamides,
h) benzoic acid,
i) dinitroanilines,
j) phosphor amidates,
k) pyridines,
I) acetamides, chloroacetamides and oxyacetamides,
m) long chain fatty acid inhibitors and inhibitors of VLCFAs,
n) tetrazolinones,
o) benzofuranes,
p) phosphoro dithioates
r) thiocarbamates, and
s) natural PGRs, such as e.g. abscisic acid, gibberellic acids, cytokinins, natural auxins and esters, ethylene releasing products.

In a preferred embodiment the one or more herbicides to be used in combination with one or more compounds of the invention are one or more soil herbicides.

The compositions according to the invention have excellent herbicidal efficacy against a broad spectrum of economically important monocotyledonous and dicotyledonous annual harmful plants. The compositions act efficiently even on perennial harmful plants which produce shoots from rhizomes, root stocks and other perennial organs which are difficult to control.

Specific examples may be mentioned of some representatives of the monocotyledonous and dicotyledonous weed flora which can be controlled by the compositions according to the invention, without the enumeration being restricted to certain species. Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Eleteranthera, Imperata, Ischaemum, Leptochloa, Folium, Monochoria, Panicum, Paspalum, Phalaris, Phleum, Poa, Rottboellia, Sagittaria, Scirpus, Setaria, Sorghum.

Dicotyledonous weeds of the genera: Abutilon, Amaranthus, Ambrosia, Anoda, Anthemis, Aphanes, Artemisia, Atriplex, Beilis, Bidens, Capsella, Carduus, Cassia, Centaurea, Chenopodium, Cirsium, Convolvulus, Conyza, Datura, Desmodium, Emex, Erigeron, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Ulex, Urtica, Veronica, Viola, Xanthium.

The compounds of the invention are also effective against weeds from the division Teridophyta, like horsetail (equisetum) or bracken.

The compositions of the invention are also efficient against moss. Specific examples may be mentioned of some representatives of the mosses which can be controlled by the compositions according to the invention, without the enumeration being restricted to certain species: Polytrichum commune, Tortula muralis, Hypnum cypressiforme, Grimmia pulvinata, Calliergonella cuspidate, Pseudoscleropodium purum, Brachythecium rutabulum, Rhytidiadelphus triquetrus and Rhytidiadelphus squarrosus.

The compositions of the invention can also be used as effective for the removal of green algae, lichen, mould and fungal stains from all kinds of hard surfaces, including concrete, brick paving, patios, paths, fences, sheds, greenhouse and conservatory glass.

The compounds of the invention are particularly useful for burn-down applications.

By virtue of their herbicidal and plant-growth-regulatory properties, the compositions of the invention can also be employed for controlling harmful plants in crops of genetically modified plants or plants modified by conventional mutagenesis. In general, the transgenic plants are distinguished by especially advantageous properties, for example by resistances to certain pesticides, mainly certain herbicides, resistances to plant diseases or causative organisms of plant diseases, such as certain insects or microorganisms such as fungi, bacteria or viruses. Other specific characteristics relate, for example, to the harvested material with regard to quantity, quality, storability, composition and specific constituents. Thus, transgenic plants are known whose starch content is increased, or whose starch quality is altered, or those where the harvested material has a different fatty acid composition.

For proper use in transgenic crops today one of ordinary skill in the art could determine an appropriate application dosage, which may vary with crop, objective weeds, and weather conditions and so on. Future breeding programs may give rise to development of transgenic crops with germ plasm with resistance to pelargonic acid.

The compositions of the present invention may be utilized without modification or may be diluted with water to give a solution or an emulsion and applied to weeds.

As products, the inventive compositions are in a concentrated form whereas the end-user generally employs diluted compositions but application as concentrate is also possible. Said compositions may be diluted to concentrations down to 1.0 to 20% of the herbicidal ester and more preferably 1-10% and most preferably 3 to 10% of herbicidal ester. The doses usually are in the range of about 5 to 200 kg a.i./ha, preferably 5 to 100 kg a.i./ha, and most preferably 5 to 50 kg a.i./ha.

One of ordinary skill in the art could determine an appropriate application dosage, which may vary with crop, objective weeds, and weather conditions and so on.

The invention therefore also provides a method of controlling unwanted vegetation, preferably in crops of plants, where one or more compound(s) according to the invention is/are applied to the plants (for example harmful plants such as monocotyledonous or dicotyledonous weeds or undesired crop plants), to the seeds (for example grains, seeds or vegetative propagules such as tubers or shoot parts with buds) or to the area on which the plants grow (for example the area under cultivation). In this context, the compounds according to the invention can be applied for example post-emergence, pre-emergence or pre-sowing (if appropriate also by incorporation into the soil).

The invention therefore also provides methods for sucker control, desiccation and defoliation, chemical pruning, e.g. flower (blossom) thinning applications in orchards and pinching in ornamentals and vegetables by applying one or more compounds of the invention.

For a clearer understanding of the invention, specific examples are set forth below. These examples are merely illustrations and are not to be understood as limiting the scope and underlying principles of the invention in any way. Indeed, various modifications of the invention in addition to those shown and described herein will become apparent to those skilled in the art from the following examples and foregoing description. Such modifications are also intended to fall within the scope of the appended claims.

### EXAMPLES

The percentages stated hereinafter are percent by weight (% by weight), unless explicitly stated otherwise.

The raw materials used are:

| | |
|---|---|
| Vorox | Commercial pelargonic acid herbicide formulation, |
| | Compo |
| Water | deionized water or tap water |
| MCPA | MCPA auxin herbicide >99% purity, Sigma aldrich |
| Dicamba | Dicamba auxin herbicide >98% purity, Schirm |
| Pelargonic acid | Pelargonic acid >99% purity, Novamont |
| Genagen PA | Nonanoy dimethyl amide solvent, Clariant |
| Emulsogen EL | Castor oil ethoxylate, Clariant |
| 400 | |
| Hostaphat 1306 | Phosphoric ester of alcohol ethoxylate, Clariant |
| Aclonifen | Aclonifen soil herbicide, Sigma Aldrich |
| Abscisic acid | Abscisic acid plant growth regulator, Sigma Aldrich |
| Diflufenican | Phenoxynicotinanilide soil herbicide, Sigma Aldrich |
| Flufenacet | Oxyacetamide soil herbicide, Sigma Aldrich |
| Cloquintocet- | Safener, Sigma Aldrich |
| mexyl | |

### Example 1: Preparation of the Fatty Acid Compounds of the Invention

The inventive compounds are listed in Table 1. All test substances were liquid, which makes them easy to handle and pourable.

TABLE 1

| [symbol] scription | R1 | R2 | R3 | m | R4 | R5 | n | R6 | R7 | p |
|---|---|---|---|---|---|---|---|---|---|---|
| [symbol] ester methyl ether | linear C8-alkyl | H | H | 6 | — | — | 0 | — | — | 0 |
| [symbol] ester methyl ether | linear C8-alkyl | H | H | 8 | — | — | 0 | — | — | 0 |
| [symbol] 6 EO ester methyl ether | linear C8-alkyl | H | H | 4 | CH₃ | H | 1 | H | H | 2 |
| [symbol] 2 EO ester methyl ether | linear C8-alkyl | — | — | 0 | CH₃ | H | 1 | H | H | 2 |
| [symbol] EO ester methyl ether | linear C7/C9-alkyl | H | H | 4 | — | — | 0 | — | — | 0 |
| [symbol] EO ester methyl ether | linear C7/C9-alkyl | H | H | 6 | — | — | 0 | — | — | 0 |
| [symbol] EO ester methyl ether | linear C11/C13-alkyl | H | H | 3 | — | — | 0 | — | — | 0 |
| [symbol] EO ester methyl ether | linear C11/C13-alkyl | H | H | 5 | — | — | 0 | — | — | 0 |
| [symbol] ester decyl ether | linear C8-alkyl | H | H | 5 | — | — | 0 | — | — | 0 |
| [symbol] ester hexyl ether | linear C8-alkyl | H | H | 3 | — | — | 0 | — | — | 0 |
| [symbol] ester methyl ether | linear C9 alkenyl | H | H | 6 | — | — | 0 | — | — | 0 |
| [symbol] 3 EO ester methyl ether | linear C11 alkenyl | H | H | 3 | — | — | 0 | — | — | 0 |
| [symbol] oic acid | branched C9-alkyl | H | H | 6 | — | — | 0 | — | — | 0 |
| [symbol] O ester methyl ether | linear C11-alkyl | H | H | 4 | — | — | 0 | — | — | 0 |
| [symbol] EO ester methyl ether | linear C10-alkyl | H | H | 6 | — | — | 0 | — | — | 0 |
| [symbol] O ester ethyl ether | linear C8-alkyl | H | H | 6 | — | — | 0 | — | — | 0 |
| [symbol] diester | | | | | | | | | | |
| [symbol] diester | | | | | | | | | | |

*non-inventive examples [symbol]
indicates data missing or illegible when filed

### General Procedure for the Synthesis of Alcohol Ethoxylate Esters (A1-A16)

Alcohol ethoxylates were synthesized according to standard alkoxylation procedures as described in (e.g. US2012/310004). In a flask, equipped with a Dean-Stark-head, alcohol ethoxylates or glycerol were mixed with the respective carboxylic acid at a stoichiometric mixture, a catalytic amount of sulfuric acid was added and the mixture was heated up to 200° C. upon stirring under a constant stream of nitrogen. Reaction progress was followed by water separation and acid value. The final product was characterized by NMR spectroscopy and titration methods.

General procedure for the synthesis of non-inventive (poly) alcohol fatty acid diesters (A17, A18) In a flask, equipped with a Dean-Stark-head, a carboxylic acid alkoxylate and the respective carboxylic acid were mixed stoichiometric ratio of 1:1. A catalytic amount of sulfuric acid was added and the mixture was heated up to 200° C. upon stirring under a constant stream of nitrogen.

The progress of the reaction was monitored by water separation and acid value titration. The final product was characterized by NMR spectroscopy and titration methods.

### Example 2: Formulations with Pelargonic Acid 6 EO Ester Methyl Ether (A1)

The formulations have been prepared by mixing the different components listed in Table 2 to obtain homogeneous solutions.

TABLE 2
Formulations with PA derivatives
(the percentages are % by weight)

| B1 | B2 | B3 | B4 | B5 | B6 | B7 | B8 | B9 | [symbol] |
|---|---|---|---|---|---|---|---|---|---|
| 81 | 44 | 97 | 99 | 99 | 90 | 50 | 70 | 95 | [symbol] |
| 9 | 44 | | | | | | | | |
| | | 3 | 1 | | | | | | |
| | | | | 1 | | | | | |
| | | | | | 10 | 50 | | | |
| | | | | | | | 30 | | |
| | | | | | | | | 5 | |
| 10 | 8 | | | | | | | | |
| | 4 | | | | | | | | |
| [symbol] | clear, | clear, | clear, | clear, | clear, | clear, | clear, | clear, | [symbol] |
| [symbol] | stable | stable | stable | stable | stable | stable | stable | stable | [symbol] |

[symbol]

indicates data missing or illegible when filed

In all cases transparent liquid formulations were obtained which were stable over several weeks upon storage. In many cases e.g. B3-B12 the inventive compounds act as good solubilizers for active ingredients which facilitates the preparation of co-formulations with other active ingredients.

### Example 3: Production of Spray Liquids

The inventive compounds were used to make spray liquids at relevant use concentration. These spray liquids are produced by mixing the various test substances in water, and the appearance and stability of the spray liquid were assessed after 24 h.

TABLE 3

| Test | | Amount | | |
|---|---|---|---|---|
| substance | Description | [% by wt.] | Appearance | Stability |
| | Pelargonic acid | 0.3 | 2 clear phases | phase |
| | | | | separation |
| | Pelargonic acid | 5.0 | 2 clear phases | phase |
| | | | | separation |
| | Vorox | 13.0 | Cloudy | stable |
| | | | homogeneous | |
| A1 | Pelargonic acid | 0.3 | Slightly cloudy | stable |
| | 6 EO ester | | homogeneous | |
| | methyl ether | | emulsion | |
| A1 | Pelargonic acid | 5.0 | Clear solution | stable |
| | 6 EO ester | | | |
| | methyl ether | | | |
| A2 | Pelargonic acid | 0.3 | Clear solution | stable |
| | 8 EO ester | | | |
| | methyl ether | | | |
| A2 | Pelargonic acid | 5.0 | Clear solution | stable |
| | 8 EO ester | | | |
| | methyl ether | | | |
| A5 | C8/C10 fatty | 0.3 | Slightly cloudy | stable |
| | acid 4 EO ester | | homogeneous | |
| | methyl ether | | emulsion | |
| A5 | C8/C10 fatty | 5.0 | cloudy | stable |
| | acid 4 EO ester | | homogeneous | |
| | methyl ether | | emulsion | |
| A6 | C8/C10 fatty | 0.3 | Slightly cloudy | stable |
| | acid 6 EO ester | | homogeneous | |
| | methyl ether | | emulsion | |
| A6 | C8/C10 fatty | 5.0 | Clear solution | stable |
| | acid 6 EO ester | | | |
| | methyl ether | | | |

The inventive compounds can be used as 100% substances without need for additional additives or formulation aids to obtain stable spray liquids. They are either water soluble and form clear homogeneous spray liquid or self-emulsifying in the spray liquid and form cloudy homogeneous emulsions that are stable over 24 h. By contrast, spray liquids comprising pelargonic acid at similar amount phase separate immediately. Stable spray liquids can only be achieved with pelargonic acid when they are formulated with appropriate emulsifiers and other formulation aids like in the commercial benchmark Vorox. Consequently the formulated products always comprise an active substance content of pelargonic acid much lower than 100% (e.g. Vorox exhibits 25% pelargonic acid content).

### Example 3: Results of Greenhouse Trials to Test Herbicidal Activity

Standard post emergence herbicide application procedures were used, as described below, to apply inventive compounds and formulations listed in the tables 1 and 2, as well as the reference materials.

Seed of monocotyledonous and dicotyledonous harmful plants such as Abutilon theophrasti (ABUTH), Alopecurus myosuroides (ALOMY), Amaranthus retroflexus (AMARE), Digitaria Sanguinalis (DIGSA), Erigeron canadensis (ERICA), Lolium perenne (LOLPE), Solanun nigrum (SOLNI), Viola arvensis (VIOAR) were sowed in 18 cm² pots. The plants were placed in a greenhouse under controlled environmental conditions, and sub-irrigation. About one week after emergence, seedlings were thinned as needed, including removal of any unhealthy or abnormal plants, to create a uniform series of test pots.

The plants were maintained for the duration of the test in the greenhouse, where they received a mean of 70 μmol m⁻² s⁻¹ of light per day/night. Temperatures averages about 24° C. during the day and about 20° C. during the night. Plants were sub-irrigated throughout the test to ensure adequate soil moisture levels.

Pots were assigned to different treatment in a randomized experimental design. A set of pots was left untreated as a reference against which effects of the treatments could later be evaluated. Applications of tested formulations were made in a spray cabin model SPK B CT02 designed by CheckTec using the following parameters:
nozzle Lechler LU-120-08, 1000 L/ha, 3 bars, 0.56 m/s
nozzle Lechler LU-120-06, 500 L/ha, 3 bars, 0.7 m/s.

The distance of the nozzle from the plants was between 50 to 53 cms.

After treatment, pots were returned to the greenhouse until ready for evaluation (6 hours after treatment (6HAT), 1 day after treatment (1 DAT), and 2 days after treatment (2DAT).

For evaluation of herbicidal effectiveness, all plants in the test were examined by a single technician, who recorded percent control, a visual measurement of the effectiveness of each treatment by comparison with untreated plants. Control of 0% indicates no effect, and control of 100% indicates that all of the plants are completely dead. The reported % control values represent the average for all replicates of each treatment.

### Description of Effectiveness Against Weeds

| Descriptor | Control | Substance |
|---|---|---|
| Highly effective | 70-100% | A1, A4, B1-B6 |
| Effective | 50-69.9% | A2, A3, A5-A10, |
| | | A13, A14 |
| Moderately | 20-49.9% | A11, A12, A15 |
| effective | | |
| Not effective | 0-19.9% | A17, A18 |

Test substances according to the invention such as, for example, the compounds A1, A2, A3, A4, A5, A6, A7, A8, A9, A10, All, A12, A13, A14, A15, A16 and the formulations B1, B2, B3, B4, B5, B6 from the above tables 1 to 2 and, as benchmarks, pelargonic acid as well as the commercial formulation Vorox, show a herbicidal activity from moderately effective to highly effective against a plurality of the harmful plants at an application rate of 5-20% w/v of applied substances per hectare in a spray volume of 500-1000 liters per hectare when applied by broadcast foliar method, in particular against one, two, three, four, five, six, seven, or even all of the harmful plants selected from the group consisting of ABUTH, ALOMY, AMARE, ERICA, DIGSA, LOLPE, SOLNI and VIOAR.

Further test substances according to the invention such as, for example, the compound A1 and A4 and the formulations B1, B2, B3, B4, B5, B6 from the above tables 1 to 2 and also the commercial formulation Vorox, show a “highly effective” herbicidal activity against several, in particular three or more, of the mentioned harmful plants when applied by broadcast foliar method at an application rate of 5-20% w/v of applied substances per hectare in a spray volume of 500-1000 liters per hectare when applied by broadcast foliar method.

Generally, the compounds according to the invention displayed particularly similar or better herbicidal activity than the standard products in post-emergence application method against several harmful plants selected from the group, ALOMY, AMARE, DIGSA, SOLNI and VIOAR.

### Example 4: Volatility Measurements

The volatility of the test substances from the spray deposit was measured time dependent. All measurements have been carried out using a concentration of 50 g/L of the test substances in the application solution. The reference pelargonic acid was used at a concentration of 30 g/L and the commercial benchmark Vorox was applied at a concentration of 130 g/L. The pH drop volumes of 10 μl of the spray liquids which amounts to 500 μg of test substance were then applied on two different substrates. An inert wettable synthetic substrates and non-permeable leaf cuticles were used as substrates. There were 3-5 repetitions per substrate, treatment and time, respectively. After evaporation of drop water, the substrates were weighed to obtain the initial weight and placed under a sealed box with defined air suction output volume of 30 m³/h. The volatility has been determined after the desired measurement time of 4 h, 24 h and 48 h by weighing of the substrates to obtain the remaining weight of the test substances. The fraction of remaining test substance in % is obtained by the ratio of remaining test substance compared to the initial weight of test substance. The volatilized amount was calculated by subtraction of the remaining test substance of the initial applied amount. The volatilized fraction of test substance in % is obtained by the ratio of volatilized test substance compared to the initial weight of test substance. A substrate without test substance is used as reference to account for changes in environmental conditions such as humidity. Concentrations used are based on ca. 3% pelargonic acid equivalent

TABLE 4
Volatilized fraction measured on non-permeable leaf cuticles

| Test | | | Volatilized | Volatilized | Volatilized |
|---|---|---|---|---|---|
| sub- | | | fraction | fraction | fraction |
| stance | | pH | 4 h | 24 h | 48 h |
| | Pelargonic acid | 4 | 55.7 | 78.9 | 80.6 |
| | Pelargonic acid | 7 | 29.2 | 33.8 | 35.3 |
| | Vorox | 4 | 21.8 | 46.0 | 49.9 |
| | Vorox | 7 | 13.0 | 29.4 | 33.9 |
| A1 | Pelargonic acid | 4 | 1.5 | 4.8 | 5.2 |
| | 6 EO ester | | | | |
| | methyl ether | | | | |
| A2 | Pelargonic acid | 4 | 1.7 | 2.1 | 2.8 |
| | 8 EO ester | | | | |
| | methyl ether | | | | |
| A9 | Pelargonic acid | 4 | 8.6 | 11.7 | 22.8 |
| | 5 EO ester | | | | |
| | decyl ether | | | | |
| A10 | Pelargonic acid | 4 | 6.2 | 14.7 | 18.9 |
| | 3 EO ester | | | | |
| | hexyl ether | | | | |
| A17 | Pelargonic acid | 4 | 0.6 | 1.5 | 0.6 |
| | 6 EO diester | | | | |
| A18 | Pelargonic acid | 4 | 0.6 | 1.7 | 4.0 |
| | 8 EO diester | | | | |

TABLE 5
Volatilized fraction measured on inert wettable synthetic substrates

| Test | | | Volatilized | Volatilized | Volatilized |
|---|---|---|---|---|---|
| sub- | | | fraction | fraction | fraction |
| stance | | pH | 4 h | 24 h | 48 h |
| | Pelargonic acid | 4 | 3.9 | 19.1 | 35.2 |
| | Pelargonic acid | 7 | 7.3 | 21.3 | 27.9 |
| | Vorox | 4 | 3.9 | 13.6 | 22.7 |
| | Vorox | 7 | 1.7 | 7.1 | 12.0 |
| A1 | Pelargonic acid | 4 | −0.2 | 0.6 | 1.2 |
| | 6 EO ester | | | | |
| | methyl ether | | | | |
| A2 | Pelargonic acid | 4 | −0.3 | 0.2 | 0.4 |
| | 8 EO ester | | | | |
| | methyl ether | | | | |
| A9 | Pelargonic acid | 4 | 0.3 | 1.2 | 1.9 |
| | 5 EO ester | | | | |
| | decyl ether | | | | |
| A10 | Pelargonic acid | 4 | 0.9 | 4.4 | 7.2 |
| | 3 EO ester | | | | |
| | hexyl ether | | | | |
| A17 | Pelargonic acid | 4 | 0.6 | 1.0 | 1.3 |
| | 6 EO diester | | | | |
| A18 | Pelargonic acid | 4 | 0.5 | 0.7 | 0.9 |
| | 8 EO diester | | | | |

The volatility of the inventive compounds is drastically reduced for both measured substrates compared to the pelargonic acid and the commercial benchmark product Vorox, even when compared to data at neutral pH.

The lower volatility of the inventive compounds also results in a reduced smell compared to pelargonic acid and the commercial benchmark product Vorox.

### Example 5: Results for Test Plant Growth Regulator Activity for Blossom Thinning in Orchards

Fruit trees were sprayed with substance A1 (Pelargonic acid 6 EO ester methyl ether), several days before the expected full blossoming in the greenhouse, and also in a field trial to test the blossom thinning, which is used to increase the fruit production. Different fruit trees and varieties were chosen for each trial to demonstrate that the method of the present invention is applicable to a wide range of fruits trees and varieties such as Fuji, Tsugaru, Starking Delicious, Jona Gold, Mutsu, Ourin, Kougyoku, Asahi, Senshu, Red Delicious, Golden Delicious, Granny Smith, Jonathan Rome Beauty, Yellow Newton, Baldwin, Cortland, Grimes and McIntosh apple trees.

Plots (3 trees per plot in field trials; 1 tree per plot in greenhouse) were assigned to different treatments in a randomized experimental design. One plot was left untreated as a reference against which effects of the treatments could later be evaluated.

The test substance A1 was diluted in water in a range of 1-10% and the spray solution was sprayed in an amount of about 2 to about 15 liters per apple tree (height: about 2-2.5 m, width: about 1-1.5 m). The Application was made with a high-pressure power backpack sprayer manufactured by SOLO® Kleinmotoren GmbH.

The treated trees were evaluated regarding flower thinning effect, but also regarding undesired effects by chemical injury such as leaf browning, leaf malformation, etc.

Test substance A1 showed excellent results as flower thinning agent by leading to removal of flowers or interrupting the reproductive part of the flowers without significantly harming the tree, avoiding major injuries to leaves and branches when evaluated after treatment.

1.-15. (canceled)

16. A method for controlling unwanted vegetation comprising the step of applying to the unwanted vegetation a composition comprising one or more fatty acid derivatives of the formula (I) 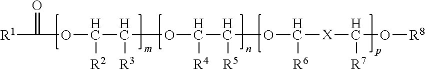
wherein
R¹ is an aliphatic group containing 5 to 15 carbon atoms, wherein the aliphatic group is linear or branched and is saturated or unsaturated;
R², R³, R⁴, R⁵, R⁶, R⁷ are, independently, hydrogen or methyl;
m, n, and p are numbers from 0 to 15, wherein
1≤m+n+p<18;
the different monomers can be arranged in statistical order, alternatingly, or as a block copolymer; and m, n, and p can be a statistical mixture;
X is, independently, a covalent bond or hydroxy methylene; and
R⁸ is an aliphatic group containing 1 to 4 carbon atoms, wherein the aliphatic group is linear, branched, or cyclic, and is saturated or unsaturated.

17. The method according to claim 16, wherein R¹ is a linear or branched alkyl group containing 7 to 11 carbon atoms.

18. The method according to claim 16, wherein R¹ is n-octyl.

19. The method according to claim 16, wherein n is 0 and p is 0.

20. The method according to claim 16, wherein R² is H and R³ is H.

21. The method according to claim 16, wherein m is a number from 1 to 10.

22. The method according to claim 16, wherein m is a number from 3 to 7.

23. The method according to claim 16, wherein the repeating units (OCHR²CHR³), (OCHR⁴CHR⁵), and (OCHR⁶CHR⁷) are, independently, ethylene glycol or 1,2-propylene glycol.

24. The method according to claim 16, wherein
R¹ is an alkyl group containing 5 to 13 carbon atoms, wherein the alkyl group is linear or branched;
m is a number from 0 to 15, n is a number from 1 to 5, p is a number from 1 to 5; the different monomers are arranged in statistical order, alternatingly, or as a block copolymer; and m, n, and p are a statistical mixture;
X is a covalent bond; and
R⁸ is a methyl group.

25. The method according to claim 16, wherein the composition comprises at least one fatty acid derivative of the formula (I) wherein R¹ is a linear C7 alkyl and at least one fatty acid derivative of the formula (I) wherein R¹ is a linear C9 alkyl.

26. The method according to claim 16, wherein
R¹ is octyl;
R² and R³ are H;
n and p are 0;
m is a number greater than 4 and less than 12; and
R⁸ is methyl.

27. The method according to claim 16, wherein
R¹ is octyl;
R² and R³ are H;
n and p are 0;
m is 6; and
R⁸ is methyl.

28. The method according to claim 16, wherein the composition comprises from 2 wt % to 99 wt % of the one or more fatty acid derivatives of the formula (I), based on the total weight of the composition.

29. The method according to claim 16, wherein the composition consists of the one or more fatty acid derivatives of the formula (I).

30. The method according to claim 16, wherein the composition further comprises one or more herbicides, plant growth regulators, or combinations thereof.

31. The method according to claim 16, wherein the composition further comprises between 10 wt-% and 50 wt-% of one or more organic solvents, based on the total weight of the composition.

32. The method according to claim 16, wherein the composition does not contain an organic solvent.

33. A composition comprising one or more fatty acid derivatives of the formula (I) 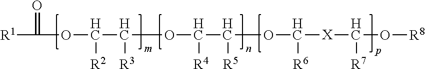
wherein
R¹ is an aliphatic group containing 5 to 15 carbon atoms, wherein the aliphatic group is linear or branched and is saturated or unsaturated;
R², R³, R⁴, R⁵, R⁶, R⁷ are, independently, hydrogen or methyl;
m, n, and p are numbers from 0 to 15, wherein
1≤m+n+p<18;
the different monomers can be arranged in statistical order, alternatingly, or as a block copolymer; and m, n, and p can be a statistical mixture;
X is, independently, a covalent bond or hydroxy methylene; and
R⁸ is an aliphatic group containing 1 to 4 carbon atoms, wherein the aliphatic group is linear, branched, or cyclic, and is saturated or unsaturated.

34. The composition according to claim 33, wherein
R¹ is octyl;
R² and R³ are H;
n and p are 0;
m is a number greater than 4 and less than 12; and
R⁸ is methyl.

35. The composition according to claim 33, wherein
R¹ is octyl;
R² and R³ are H;
n and p are 0;
m is 6; and
R⁸ is methyl.
